(19) United States
(12) Patent Application Publication
(10) Pub. No.: US 2005/0278014 A9
(43) Pub. Date: Dec. 15, 2005

(54) Stent and method for drug delivery from stents

(76) Inventors: Wolfgang Daum, Groton, MA (US); Monica L.B. Anderson, Plymouth, MN (US); Rudy A. Mazzocchi, Indian Harbour Beach, FL (US)

Correspondence Address: PEPPER HAMILTON LLP, ONE MELLON CENTER, 50TH FLOOR, 500 GRANT STREET, PITTSBURGH, PA, 15219, US

(21) Appl. No.: 10/078,622

(22) Filed: Feb. 19, 2002

Related U.S. Application Data
(63) Continuation-in-part of application No. 10/044,475, filed on Jan. 10, 2002, now Pat. No. 6,786,904.
(60) Provisional application No. 60/273,850, filed on Mar. 7, 2001.

(51) Int. Cl. A61F 2/06

(52) U.S. Cl. 623/1.15; 623/1.42; 604/891.1

(57) ABSTRACT

A method for controlling the activity of drugs on or in drug-coated or drug-loaded implantable devices, such as stents or other metallic devices, uses non-invasive, inductive heating of such device. The heating of a device, such as stent, can be used to release drugs applied to the stent in release layers, to activate drugs on the stent that have little or no activity at body temperature and to enhance for defined periods the reaction environment at the stent for drug-adjacent tissue interactions. Reverse effects of deactivation of drugs upon heating are also possible.

### CROSS REFERENCE TO RELATED PATENT APPLICATION

This application claims priority from the provisional patent application, Serial Number 60/273,850, filed Mar. 7, 2001, and the subject matter of which is incorporated herewith by reference.

### TECHNICAL FIELD

The present invention relates to implantable devices, such as stents, used for implantation in tissue for cardiovascular intervention and other purposes and the delivery of drugs placed on or in the stent. In particular, the present invention relates to a stent prepared to deliver drugs when heated by electromagnetic fields and a method and system for causing drug-coated or drug-loaded stents to deliver their drugs into the blood stream of a cardiovascular vessel or into surrounding tissue.

### BACKGROUND OF THE INVENTION

Different techniques are known to prevent in-stent restenosis of cardiovascular or other stents. In-stent restenosis affects nearly 50% of all stenting procedures. Known techniques to prevent in-stent restenosis are the use of radioactive stents (brachytherapy), biodegradable stents, drug-coated stents and inductive heating of stents.

Stents can be coated or loaded with different drug formulations, including materials such as biologically active micro-spheres used for controlled release of biologically active agents inhibiting restenosis of the stent. These drugs can be included in encapsulations such as polyethylene glycol substances that are formulated to dissolve within a period of time to release the biologically active micro spheres into the vessel wall of the organ or the vessel in which the stent is located.

One problem with these drug-coated and drug-loaded stents is that the dissolving or eluting mechanism of the drug is not controllable or selectable by the physician. Whatever time release is designed into the drug coating or loading, together with conditions within the patient, will cause the drug to be delivered in a manner that cannot be controlled or selected once the coated or loaded stent is inserted. Thus, the drug effect will continue to run its course. If the drug is designed to have an inhibiting effect on tissue growth, that effect may go too far and actually be deleterious to the tissue. This problem is addressed by this invention.

### SUMMARY OF THE INVENTION

An object of the present invention is to provide a mechanism for controlling the delivery or activity of a drug placed on or in a drug-delivery stent and to provide such control non-invasively from outside the patient's body. In German Gebrauschmuster DE 295 19 982.2 and in European patent application EP 1 036 574 A1 inductive or hysteresis-loss methods for heating up stents non-invasively with electromagnetic fields have been presented. The stated purpose of this heating is to prevent or retard cell growth in the regions adjacent the stent. The heating of the stent is contemplated to be sufficient to render the cells adjacent the stent non-viable.

During inductive heating as described in, e.g., patent DE 295 19 982.2 the stent heats up from normal body temperature of 37.6° C. to higher temperatures, typically above 40° C. The heat energy can then be used in several different ways to control activity of a drug that is coated on or loaded in a stent. First, the heat within a stent can be used to activate a heatsensitive drug-releasing material (e.g., a fiber) from which the stent is made. The heat thus makes available a drug that is otherwise captured within the stent material and is wholly or largely not available for activity with adjacent tissue. With a properly-selected drug-releasing material, the opposite effect is also possible, i.e., that heat deactivates the material or prevents or inhibits release. Second, the heat within the stent is conducted by thermal heat conduction to the outer surface of the stent. If a drug coating is at that surface, the heat can be used to activate a drug that is wholly or largely inactive at normal body temperatures. Alternatively, if the drug is contained in a heat-sensitive release coating that is on the stent surface, the heat energy at the stent surface can cause the drug to be released, so that it can diffused or dissolved into adjacent tissue. Again, with a properly selected drug formulation, heating to cause drug deactivation or inhibition of drug release is also possible. Third, as the heat energy at the stent surface travels by heat conduction into the tissue adjacent the stent, the proteins and other molecules in the tissue will also become heated. Thus, not only is the drug released, but the microenvironment in which the drug and adjacent tissue interact will be heated. This heating may enhance or otherwise affect the drug-tissue reactions in ways that are not present when one or both are at lower temperatures.

In one particular embodiment, the drug coated on or loaded in the stent is a restenosis-preventing drug. According to the above possibilities, the drug can be released by elevated temperatures from within or at the surface of the stent, it can be activated (or deactivated) by elevated temperatures at the stent surface and/or the drug-adjacent tissue reaction can be enhanced by elevated temperatures in the stent or at its surface and also in the adjacent tissue.

The present invention uses the stent heating method to provide control over delivery of one or more drugs from a drug-coated or drug-loaded stent. The dissolution and/or dispersion of a drug is usually a function of temperature. The higher the temperature is, the faster the drug will dissolve or disperse into the surrounding medium from the surface where it is placed. Duration of the elevated temperature also plays a role in increasing the amount of drug delivered.

According to the present invention, a stent can be made for selective drug delivery by placing the drug to be delivered onto the stent in such a way that it is encapsulated in a release layer, or the drug can be coated on the stent directly without such a layer. In the latter case, the drug on the stent is not removed from encapsulation by heating. Rather it is selected and/or formulated so that it has its active effect when it and/or the surrounding tissue is at or above an elevated threshold temperature; when the drug and/or the surrounding tissue is below the elevated threshold temperature, the drug has no active effect.

Although stents prepared with variety of drugs that can be delivered in this way are possible, one application is a stent bearing a drug that would help prevent restenosis from occurring. We propose a stent to deliver or activate a restenosis-preventing drug. The drug may be located directly on the surface of the stent or within the stent or inserted in an encapsulation layer on the surface of the stent. In all cases the stent-carried drug will not be available or be active at body temperature, but it becomes available or active at a certain temperature point above body temperature. (The reverse effect of a drug active at body temperature and selected to become inactive is also possible and may be useful.) The invention also involves a treatment method. In order to make the drug available or active at the stent surface, the stent with the drug has to be heated. The patient will come to the hospital in a defined sequence to be treated for a certain period of time with stent heating to certain temperatures selected based on the drug and/or its encapsulation and/or the drugtissue interaction at various layers. The drug then will be delivered into or at the patient's blood or vessel wall.

### BRIEF DESCRIPTION OF THE DRAWINGS

FIG. 1 is a schematic, cross-sectional view of a stent with a layer of encapsulated drug material on the stent surface.

FIG. 2 is a schematic, cross-sectional view of a stent with drug layer that is on the stent surface and not encapsulated.

FIG. 3 is a schematic, cross-sectional view of a stent with drug material captured within the stent material.

### DETAILED DESCRIPTION OF THE PREFERRED EMBODIMENTS

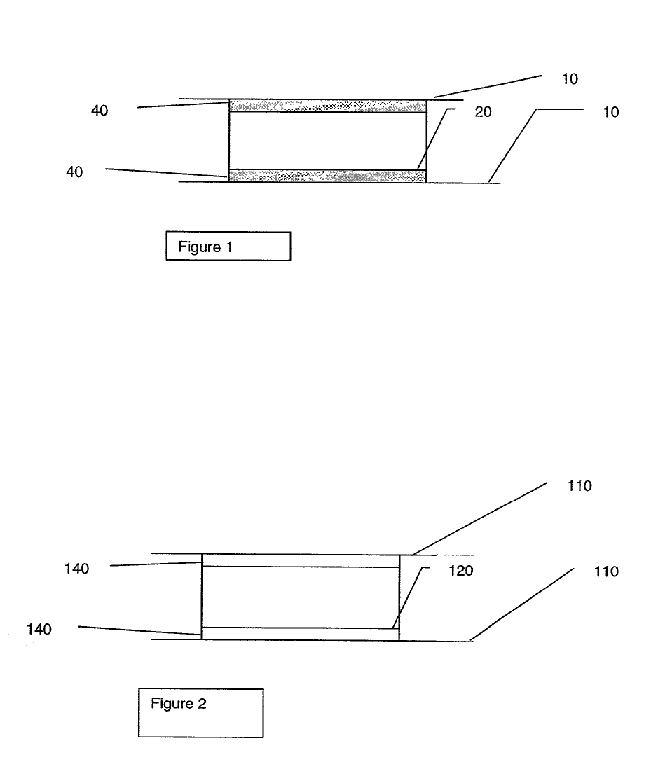

FIG. 1 shows an embodiment of the invention. A thin-walled stent 20 of generally cylindrical shape is shown inserted within tissue, where such tissue may be the interior of a blood vessel with opposing walls 10 enclosing the stent 20. On the exterior of the stent 20 is a layer of drug material 40, which is in direct contact with the tissue 10. (In reality, the stent 20 will normally be woven wires or a grid of some kind; thus, the “exterior” of the stent 20 is not solely the outer surface of the cylindrical form of the stent, but also includes other portions of the stent 20 that contact the tissue 10, whether these are on the outer surface of the cylindrical form or the inner surface or interstitial surfaces in between the two.) In this embodiment, the drug material 40 comprises an active drug dispersed in an encapsulation material that prevents the active drug from having effective contact with the tissue 10 at normal body temperatures. However, at elevated temperatures, the encapsulation material that is part of the drug material 40 breaks down to release the active drug and permit molecules of the active drug to interact with molecules of the tissue 10.

For example, the active drug can be a restenosis-preventing drug. The restenosis preventing drug is inserted into or encapsulated in a biodegradable polymer, such as a polyethylene glycol composition, to form the drug material layer 40. The stent 20 is then heated at a temperature of 39° C. and the biodegradable polymer dissolves. This makes the drug available to contact or interact with the tissue 10 surrounding the stent 20. In fact, the drug will in most cases diffuse somewhat into the surrounding tissue, thus making its active effect available not only at the exterior of the stent 20, but also at small distances therefrom. Preferably, the heating is applied non-invasively. This can be done by a radio frequency generator device that generates an electromagnetic field sufficient to cause inductive (and/or hysteresis loss) heating in the stent. Such devices are described in Gebrauchsmuster DE 295 19 982.2 and in European patent application EP 1 036 574 A1. When the inductive heating treatment is turned off, the stent 20 will cool down to normal body temperature and the heat-activated process stops. This procedure can be repeated several times. (As noted above, the opposite effect is also possible, i.e., that heat deactivates the material or prevents or inhibits release.) As long as the supply of the drug material is not exhausted, more of the encapsulation layer will break down and more of the active drug will be released.

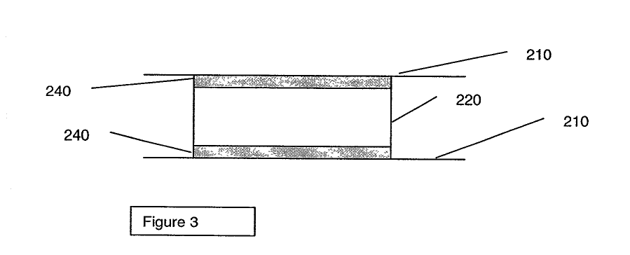

Another embodiment is shown in FIG. 2. A thin-walled stent 120 of generally cylindrical shape is shown inserted within tissue, where such tissue may be the interior of a blood vessel with opposing walls 110 enclosing the stent 120. On the exterior of the stent 120 is a layer of drug material 140, which is in direct contact with the tissue 110. (In reality, the stent 120 will normally be woven wires or a grid of some kind; thus, the “exterior” of the stent 120 is not solely the outer surface of the cylindrical form of the stent, but also includes other portions of the stent 120 that contact the tissue 110, whether these are on the outer surface of the cylindrical form or the inner surface or interstitial surfaces in between the two.) In this embodiment, the drug material 140 comprises an active drug that is formulated so that it has substantially no effect on the tissue 110 at normal body temperatures. However, at elevated temperatures, the active drug undergoes a change that makes it active. Thus, the previously substantially inert molecules of the active drug begin to interact with molecules of the tissue 110. (As noted above, with a properly selected drug formulation, heating to cause drug deactivation or inhibition of drug release is also possible.) This effect can be achieved by heating that causes changes in the activity level of either the active drug with which the stent is coated or by changes in the activity level of proteins or other molecules in the tissue 110 with respect to the active drug. That is, heating may have an effect on the reaction speed or nature of the interaction of the active drug and the tissue 110 at the drug-adjacent tissue interfaces.

A further embodiment is shown in FIG. 3. A stent 220 of generally cylindrical shape is shown inserted within tissue, where such tissue may be the interior of a blood vessel with opposing walls 210 enclosing the stent 220. The walls 240 of the stent 220 are impregnated or loaded with drug material, which is mainly not in direct contact with the adjacent tissue 210. In this embodiment, the drug-loaded walls 240 contain an active drug that is formulated into the wall material so that it has substantially no effect on the adjacent tissue 210 at normal body temperatures. However, at elevated temperatures, the active drug is released from within the walls 240. Thus, the previously substantially unavailable molecules of the active drug begin to interact with molecules of the adjacent tissue 210. This effect can be achieved by heating that causes changes in the binding of the active drug with which the stent is loaded or by actual dissolution of the walls 240 loaded with the active drug. That is, heating may have an effect on the release of the active drug from the walls 240 or the integrity of the walls 240. In either event, the heating of the stent causes increased availability of the active drug at the drug-adjacent tissue interfaces.

### EXAMPLES

The herewith claimed method of heating stents to heat a drug layer applied to the stent and heat surrounding tissue may help other drug delivery techniques to deliver their drugs in a controllable or selective way.

### EXAMPLES ARE

In U.S. Pat. No. 5,980,566 an iridium oxide coating for a stent has a biodegradable carrier of drugs applied thereto for beneficial localized action, as by incorporating into the carrier along the inward-facing surface an anticoagulant drug to reduce attachment of thrombi with blood flow through the lumen of the stent. Heat delivered through the method as claimed here could selectively enhance drug release or availability to help the process to reduce the attachment of thrombi with blood flow through the lumen of the stent.

In U.S. Pat. No. 5,980,551 (see also PCT application W098/34669) a stent has biologically active micro spheres that release a biologically active agent into the vessel wall or organ. To inhibit restenosis of the stent the biologically active micro spheres include encapsulated PGE1 in a water soluble polyethylene glycol mix. The temperature increase process as described here could help selectively control the period of time to dissolve and release the PGE1 into the vessel wall or organ.

In the U.S. Pat. No. 5,980,551 an anti-coagulation drug is incorporated into a biodegradable material to form a liquid-coating material. The temperature process as described in the present invention could help to continue this integrated coating which is less than about 100 microns.

In the application described in U.S. Pat. No. 5,733,327 the temperature elevating process described in the present invention could help selectively control the dissolution mechanism of poly-e-caprolactone, poly-D, L-deca-lactone, poly-dioxane and copolymer.

In the application described in U.S. Pat. No. 5,700,286 the process as described in the present invention could help enhance effectiveness for the lubricious material, which can be polyethylene, oxide, polyethylene glycol, polyethylene acetate, polyvinyl pyrrolidone, polyvinyl alcohol, polyacrylamide, hydrophilic soft segment urethanes, some natural gums, polyanhydrides or other similar hydrophilic polymers, and combinations thereof.

In the application described in PCT patent WO 00/56376 the temperature method as described in the present invention could help selectively degrade devices formed of polyhydroxylkanoates. These are taught as used in conjunction with metal that can be inductively heated.

In the application described in German patent application DE 197 37 021 A1 the method as described in the present invention could help selectively oxidize the medical implant which is made of magnesia, iron or zinc or other suitable materials.

In the application described in PCT application WO 96/33757 the temperature treatment of the present invention could help selectively control the process of dissolving the surface coating with a physiological acceptable polymer, such as polyvinyl alcohol or fibrinin, containing dissolved or dispersed therein a nitroso compound, such as 2-metyhyl-2-nitrosopropane.

In the application described in German patent application DE 195 14 104 A1 the method as described in the present invention could support the selective dissolution of the drug such as poly-D, L-lactide, thrombine inhibitors and other derivates.

Inductive Heating

Heating of stents as contemplated by this invention can be performed with metallic stents having adequate magnetic permeability or field absorbing qualities according to the teachings of German Gebrauchsmuster DE 295 19 982.2 and European patent application EP 1 036 574 A1. (The disclosures of these are incorporated by reference.) In these, electromagnetic fields are generated at a coil or other sending antenna and the stent is placed in the field with an orientation and at a distance and location that permit sufficient power to be absorbed at the stent (acting as a receiving antenna), such that heat can be generated in the stent. The amount of heat energy delivered to stent and the duration of heating are important variables for the drug activity selective control contemplated by this invention. The electromagnetic energy may be provided in controlled, brief pulses to permit a more precise control of the energy delivered to the stent and resulting heating effects. The greater the control of heating, the greater the control of the resulting drug release, or drug activation or drug-adjacent tissue reaction enhancement.

As used herein, a “stent” is any implantable device that provides some support or structure to surrounding tissue. Thus, the invention is applicable to a variety of stents or supporting implantable devices, not just those that are used in blood vessels. As used herein, a “drug” means a substance that has therapeutic effect, which may include gene therapy formulations as well as more conventional drugs based on chemical formulations or biological derivatives.

It is appreciated that besides stents, any other type of suitable implantable devices can be used within the scope and spirit of the present invention to controllably elute a drug off of an implantable device. Also, the implantable devices may be used just for the purpose of eluting drugs into the body. One of such implantable devices may be a metallic hip joint which is coated with a drug for better biocompatibility. The drug may be eluted by temperature. Also, a device may be made as a ball shaped type or as many small pills which are implanted just to be heated inductively to elute the drug. Thus, the invention is applicable to any implantable object (whether or not it has a prosthetic or other function) that has the ability to be heated in the manner described herein so as to cause drug release and that can be placed in a position at which or from which drug delivery is desired.

It is also appreciated that the devices can be temporarily implanted or permanently implanted. These device may be used to help chemotherapy or any other therapy.

One exemplary application can be to implant a metallic coil or pellet in the patient's prostate and use the above described invention to control the elution of a drug to treat a prostate disease. Other exemplary applications may be to control the elution of insulin off of an implantable device in a diabetic patient, or to control the elution of a drug off of an ophthalmic device in the eye to treat vision related diseases.

Accordingly, the present invention provides an implantable device having at least one coated drug material capable of being heated inductively and delivering the drug material to a body when heated. The frequency of the inductive heat is preferably below 1 MHz. Under 1 MHz, the body tissue is generally opaque for radio frequency inductive heating, above that frequency the body tissue absorbs the energy and is heated itself.

While the present invention has been described with reference to several embodiments thereof, those skilled in the art will recognize various changes that may be made without departing from the spirit and scope of the claimed invention. For example, implantable devices can be energized by inductive heating, radio or microwave frequency and tissue transmitting light technology, etc. It is noted that light of certain lower wavelength can travel further into tissue than light of a higher wavelength and, therefore, is absorbed deeper in the tissue. This effect can be used to absorb the light deeper to heat up implants deeper in the tissue. Accordingly, this invention is not limited to what is shown in the drawings and described in the specification but only as indicated in the appended claims, nor is the claimed invention limited in applicability to one type of drug. Any numbering or ordering of elements in the following claims is merely for convenience and is not intended to suggest that the ordering of the elements of the claims has any particular significance other than that otherwise expressed by the language of the claims.

1. An implant for delivery of a drug, comprising:
an implant body capable of heating by exposure to an electromagnetic field; and
a drug material applied to the implant body, said drug material being substantially effective only when the implant has been heated by exposure to the electromagnetic field and heat energy from the implant has heated the drug material.

2. The implant of claim 1, wherein the drug material is a drug ingredient combined with a heat sensitive release material, and the drug material becomes effective after the release material releases a portion of the drug ingredient.

3. The implant of claim 1, wherein the drug material is a drug ingredient adhered to the implant that is substantially inactive at normal body temperature and that becomes active after the implant has heated the drug ingredient to a temperature where is substantially active.

4. The implant of claim 1, wherein the drug material is a drug ingredient that is to be delivered to tissue adjacent the implant and drug-tissue interaction is enhanced when heat from the implant causes tissue adjacent the implant to rise above normal body temperature when the drug ingredient is present.

5. The implant of claim 1, wherein the implant is a stent and the drug material comprises an active ingredient that inhibits restenosis in the stent.

6. A method of using a drug-coated or drug-loaded implant by heating the implant above a certain temperature at which drug activity in the tissue adjacent the implant starts and maintaining that temperature for a specified period of time.

7. The method of claim 6, wherein the implant is heated by radio frequency (RF) energy.

8. The method as recited in claim 6, wherein the RF energy is generated by a sending antenna outside the patient's body transferring energy to the implant.

9. A method as cited in claim 6, wherein a sending antenna is placed inside the implant by an endovascular catheter inserted through vessels.

10. A method as recited in claim 6, wherein the drug activity is inhibiting proliferation of cells that cause restenosis.

11. An implant for delivery of a drug, comprising:
an implant body capable of heating by exposure to an electromagnetic field; and
a supply of drug material applied to the implant body, said drug material being substantially ineffective after the implant has been heated by exposure to the electromagnetic field and heat energy from the implant has heated the drug material.

12. The implant of claim 11, wherein the drug material is a drug ingredient combined with a heat sensitive release material and the drug material becomes ineffective after the release material is heated.

13. A metallic implant for delivery of a drug, comprising:
a body capable of being heated; and
a layer of drug material applied to the body, said drug material being effective while being heated.

14. A method of using a drug-coated or drug-loaded implant by heating the implant above a certain temperature at which drug activity in the tissue adjacent the implant is substantially enhanced and maintaining that temperature for a specified period of time.

15. An apparatus for delivery of a drug in a body comprising an implantable member with the drug, the member being implanted in the body and controllably heated to elute the drug off of the member to treat the body, wherein the drug is operative when the member is heated.

16. The apparatus of claim 15, wherein heating of the implantable member is invasive and is accomplished by applying a magnetic field over the body.

17. The apparatus of claim 16, wherein the elution of the drug from the implantable member is to treat prostate disease.

18. The apparatus of claim 16, wherein the elution of the drug from the implantable member is to treat diabetic disease.

19. The apparatus of claim 16, wherein the elution of the drug from the implantable member is to treat ophthalmic disease.

20. A method of delivering a drug in a body by controllably heating an implantable member with the drug to elute the drug from the member to treat the body, wherein the drug is operative when the member is heated.

21. An implantable device having at least one coated drug material capable of being heated inductively and delivering the drug material to a body when heated.

22. The device of claim 21, wherein frequency of inductive heat is below 1 MHz.

23. The device of claim 21, wherein the implantable device is a prosthetic device.
